(19) United States
(12) Patent Application Publication
(10) Pub. No.: US 2003/0216642 A1
(43) Pub. Date: Nov. 20, 2003

(54) Radiopaque and MRI compatible catheter braid

(76) Inventors: Henry J. Pepin, Loretto, MN (US); Martin R. Willard, Burnsville, MN (US); Pu Zhou, Plymouth, MN (US); Greg Kampa, Blaine, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 10/146,980

(22) Filed: May 16, 2002

(51) Int. Cl. A61B 6/00; A61M 25/00

(52) U.S. Cl. 600/431; 600/435

(57) ABSTRACT

An intravascular catheter having an elongate shaft that is entirely non-magnetically responsive and at least partially radiopaque, including a reinforcement layer. The reinforcement layer may comprise a non-magnetically responsive radiopaque metal, such as a multi-strand Tungsten braid. The improved shaft of the present invention is compatible with x-ray and MRI visualization techniques, and may be incorporated into a wide variety of intravascular catheters such as guide catheters, diagnostic catheters, balloon catheters, etc.

### FIELD OF THE INVENTION

The present invention generally relates to catheter shafts. More specifically, the present invention relates to reinforced catheter shafts for intravascular devices such as guide catheters, diagnostic catheters, balloon catheters, and the like.

### BACKGROUND OF THE INVENTION

Diagnostic catheters and guide catheters are commonly used to facilitate the diagnosis and treatment of vascular diseases such as coronary artery disease and peripheral vascular disease. Such catheters commonly include a braid reinforcement layer disposed between an inner layer and an outer layer. The braid reinforcement provides torsional rigidity, column strength, kink resistance, as well as radiopacity. However, conventional braid reinforcement materials such as stainless steel are not MRI (magnetic resonance imaging) compatible due to ferro-magnetic properties. Because different visualization techniques may be employed to facilitate intravascular navigation, it is desirable to have a catheter shaft that is both radiopaque for x-ray visualization and non-magnetically responsive for MRI compatibility.

### SUMMARY OF THE INVENTION

To address these desirable features, the present invention provides, for example, an intravascular catheter comprising a reinforced shaft that is entirely non-magnetically responsive and at least partially radiopaque. In one specific example and without limitation, the present invention provides an elongate catheter shaft that is entirely non-magnetically responsive and at least partially radiopaque, wherein the shaft includes an inner layer, an outer layer, a reinforcement layer disposed between the inner and outer layers, and a soft distal tip. The reinforcement layer may comprise a braid of non-magnetically responsive radiopaque metal wires, the outer layer may comprise a non-radiopaque flexible polymer, the inner layer may comprise a non-radiopaque lubricious polymer, and the soft distal tip may comprise a polymer loaded with a radiopaque non-magnetically responsive filler. The inner layer, the outer layer and the reinforcement layer may extend from the proximal end of the shaft to the proximal end of the distal tip, leaving the tip flexible and atraumatic.

### BRIEF DESCRIPTION OF THE DRAWINGS

FIG. 1 is a plan view of an intravascular catheter in accordance with an embodiment of the present invention, shown as a guide or diagnostic catheter;

FIG. 2 is a cross-sectional view taken along line 2-2 in FIG. 1;

FIG. 3 is a longitudinal sectional view taken along line 3-3 in FIG. 1;

FIGS. 4A-4C are fragmentary views of various braid options; and

FIG. 5 is a partially sectioned fragmentary view of the catheter shaft shown in FIG. 1.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

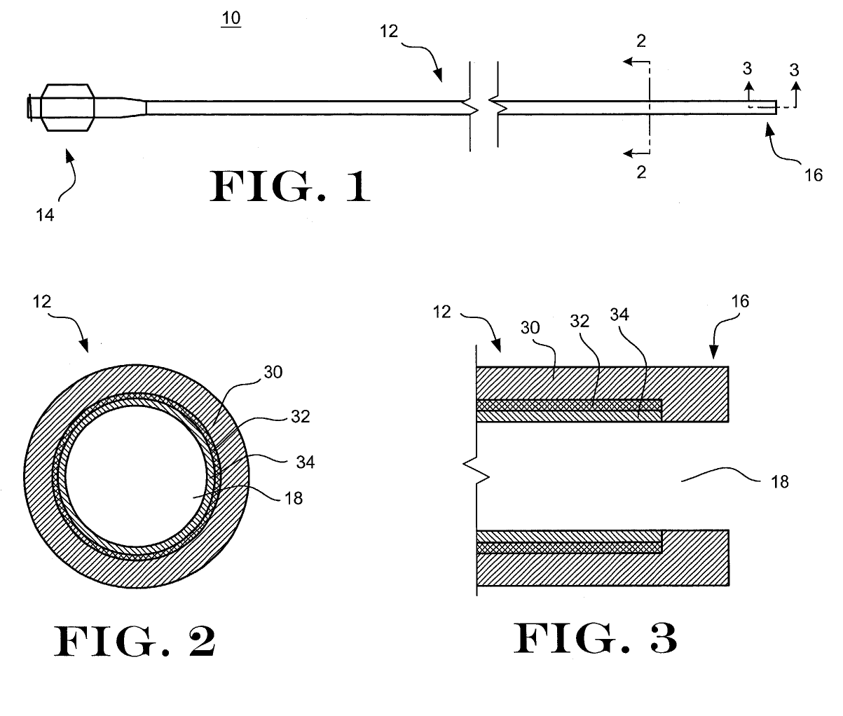

Refer now to FIG. 1 which illustrates an intravascular catheter in accordance with an embodiment of the present invention. For purposes of illustration and discussion only, the intravascular catheter shown in FIG. 1 is in the form of a guide or diagnostic catheter 10, but may comprise virtually any catheter used for intravascular applications. For example, the intravascular catheter may comprise a balloon catheter, an atherectomy catheter, a drug delivery catheter, a stent delivery catheter, etc.

As used herein, magnetically non-responsive materials refer to materials that are compatible with magnetic resonance imaging techniques. By way of example, not limitation, non-magnetically responsive materials include materials with a magnetic susceptibility (absolute value) less than 1×10⁻⁴, preferably less than 1×10⁻⁵ and ideally near zero (0). By contrast, magnetically responsive materials include materials with a magnetic susceptibility (absolute value) greater than or equal to 1×10⁻⁴ Generally speaking, polymers and some metals such as Titanium are magnetically non-responsive, and metals such as stainless steel and other ferrous containing metals are magnetically responsive.

The guide or diagnostic catheter 10 may have a length and an outside diameter sufficient to enable intravascular insertion and navigation. For example, the catheter 10 may have a length of approximately 100 cm-150 cm and an outside diameter of approximately 4F-9F. The guide or diagnostic catheter 10 may be substantially conventional except as described herein and shown in the drawings.

The catheter 10 includes an elongate shaft 12 having a proximal end and distal end. A distal tip 16 is connected to the distal end of the elongate shaft 12. The distal tip 16 and a distal portion of the elongate shaft 12 may be curved depending on the particular clinical application. The elongate shaft 12 and the distal tip 16 include a lumen 18 extending therethrough to facilitate insertion of other medical devices (e.g., guide wires, balloon catheters, etc.) therethrough, and/or to facilitate injection of fluids (e.g., radiopaque dye, saline, drugs, etc.) therethrough. A conventional manifold 14 is connected to the proximal end of the elongate shaft 12 to facilitate connection to other medical devices (e.g., syringe, Y-adapter, etc.) and to provide access to the lumen 18.

As best seen in FIGS. 2 and 3, the elongate shaft 12 may be multi-layered. In this embodiment, the elongate shaft 12 may include an outer layer 30, a reinforcement layer 32, and an inner layer 34. The distal tip 16 may comprise the outer layer 30 extending beyond the inner layer 34 and the reinforcement layer 32 to define a soft atraumatic tip.

The inner layer 34 may comprise a lubricious polymer such as HDPE or PTFE, for example. In one particular embodiment, the inner layer 34 may comprise PTFE having a wall thickness of 0.001 in., and an inside diameter of 0.058 inches. In this example, the inner layer 34 is non-magnetically responsive and non-radiopaque, but may be made radiopaque by utilizing known filler materials such as bismuth subcarbonate.

The outer layer 30 may comprise, at least in part, a polyether-ester elastomer sold under the trade name ARNITEL. The outer layer 30 may be formed, for example, by extrusion, co-extrusion, interrupted layer co-extrusion (ILC), or fusing several segments end-to-end. The outer layer may have a uniform stiffness or a gradual reduction in stiffness from the proximal end to the distal end thereof. The gradual reduction in stiffness may be continuous as by ILC or may be stepped as by fusing together separate extruded tubular segments end-to-end. The outer layer may be impregnated with a reinforcing material such as liquid crystal polymer (LCP) fibrils. For example, a proximal portion of the outer layer 30 may comprise 74D ARNITEL with 6% LCP, a mid portion may comprise 63D ARNITEL, and a distal portion may comprise 55D ARNITEL. The distal tip 16 may comprise 40D ARNITEL loaded with 46% bismuth subcarbonate to render it radiopaque. The proximal portion, mid portion, distal portion and distal tip may have lengths of 34 in., 3 in., 1.5 in., and 0.15 in., respectively. The proximal, mid and distal portions may have a wall thickness of 0.005 in., and the distal tip may have a wall thickness of 0.005 in. In this example, the outer layer 30 is non-radiopaque and non-magnetically responsive, while the distal tip 16 is radiopaque and non-magnetically responsive.

The reinforcement layer 32 may comprise a metal wire braid, for example. The metal wire braid may comprise a non-magnetically responsive (i.e., non-ferrous) radiopaque metal such as Tungsten, Gold, Titanium, Silver, Copper, Platinum, Iridium, other non-ferrous dense metals, or alloys thereof. Tungsten exhibits tensile properties (strength and rigidity) similar to or higher than those of stainless steel, which is a conventional reinforcement material that exhibits magnetic responsiveness due to some ferrous content and is therefore not MRI compatible. Tungsten is also relatively dense and therefore relatively radiopaque. Tungsten is also relatively inexpensive compared to other more precious metals and alloys.

Alternatively, the reinforcement layer 32 may be formed of a non-metal material such as poly-para-phenylene terephthalamide (KEVLAR) fibers, LCP fibers, other polymeric filaments, or glass fibers, including monofilament and multi-filament structures of each.

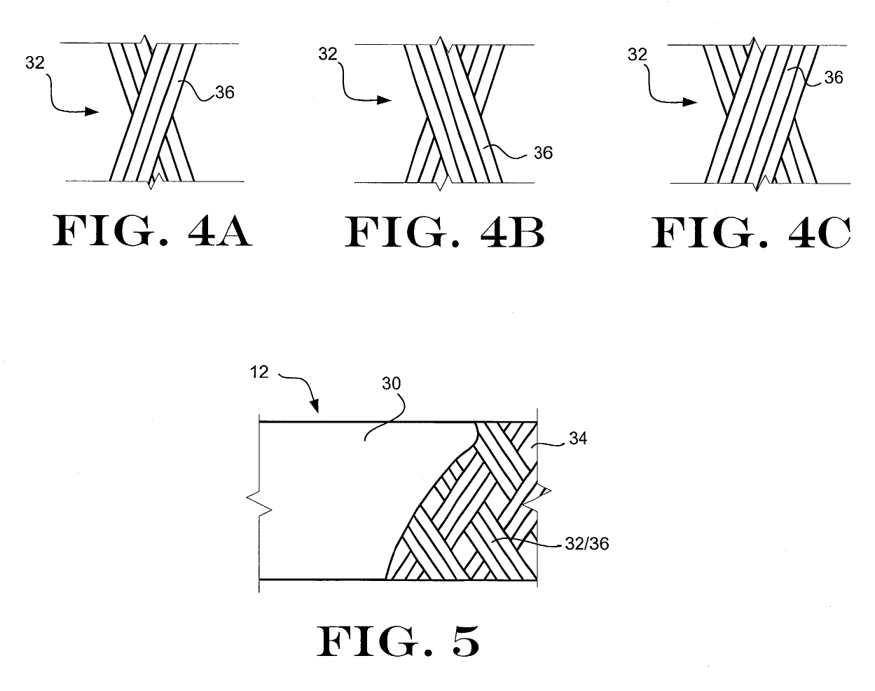

As seen in FIGS. 4A-4C, the braid reinforcement layer 32 may comprise one or more strands 36 of non-magnetically responsive (i.e., non-ferrous) radiopaque material. Each strand 36 may be flat (ribbon), round, and/or hollow. By way of example, not limitation, the braid 32 may include triple strands 36 braided in a three-over-three pattern as seen in FIG. 4A, quadruple strands 36 braided in a four-over-four pattern as seen in FIG. 4B, or quintuple strands 36 braided in a five-over-five pattern as shown in FIG. 4C. As seen in FIG. 5, a triple strand (three-over-three) reinforcement braid 32 utilizing 0.001 inch diameter Tungsten wire strands 36 with a pic count (pic count refers to the number intersections between strand sets per lineal unit) of 66+/−5 pics/inch has been found to provide good radiopacity without requiring loading of the outer layer 30, and good shaft 12 performance in terms of kink resistance, torque transmission, pushability, and shape retention.

Those skilled in the art will recognize that the present invention may be manifested in a variety of forms other than the specific embodiments described herein. Accordingly, departures in form and detail may be made without departing from the scope and spirit of the present invention as described in the appended claims.

What is claimed is:

1. An intravascular catheter comprising an elongate shaft that is entirely non-magnetically responsive and at least partially radiopaque, the elongate shaft including a reinforcement layer comprising non-magnetically responsive radiopaque metal wires.

2. An intravascular catheter as in claim 1, wherein the elongate shaft has a magnetic susceptibility (absolute value) less than 1×10⁻⁴.

3. An intravascular catheter as in claim 1, wherein the elongate shaft has a magnetic susceptibility (absolute value) less than 1×10⁻⁵.

4. An intravascular catheter as in claim 1, wherein the elongate shaft has a magnetic susceptibility (absolute value) near zero (0).

5. An intravascular catheter as in claim 2, wherein the elongate shaft includes an inner layer and an outer layer with the reinforcement layer disposed therebetween, the outer layer comprising a non-radiopaque polymer and the inner layer comprising a non-radiopaque lubricious polymer.

6. An intravascular catheter as in claim 5, wherein the elongate shaft includes a soft distal tip comprising a polymer loaded with a radiopaque non-magnetically responsive filler.

7. An intravascular catheter as in claim 6, the reinforcement layer comprises a wire braid.

8. An intravascular catheter as in claim 7, wherein the reinforcement layer consists of a non-magnetically responsive radiopaque metal.

9. An intravascular catheter as in claim 7, wherein the braid comprises multiple stands.

10. An intravascular catheter as in claim 9, wherein the multiple strands are braided in a three-over-three pattern.

11. An intravascular catheter as in claim 9, wherein the multiple strands are braided in a four-over-four pattern.

12. An intravascular catheter as in claim 9, wherein the multiple strands are braided in a five-over-five pattern.

13. An intravascular catheter as in claim 6, wherein the inner layer, the outer layer and the reinforcement layer extend from a proximal end of the shaft to the distal tip.

14. An intravascular catheter comprising an elongate shaft that is entirely non-magnetically responsive and at least partially radiopaque, the elongate shaft including a reinforcement layer consisting of a non-magnetically responsive radiopaque metal.

15. An intravascular catheter comprising an elongate shaft that is entirely non-magnetically responsive and at least partially radiopaque, the elongate shaft including a reinforcement layer consisting of a Tungsten braid.

16. An intravascular catheter comprising an elongate shaft that is entirely non-magnetically responsive and at least partially radiopaque, the elongate shaft including a reinforcement layer comprising a non-magnetically responsive radiopaque metal.

17. An intravascular catheter as in claim 16, wherein the reinforcement layer consists of a non-magnetically responsive radiopaque metal.

18. An intravascular catheter as in claim 16, wherein the non-magnetically responsive radiopaque metal comprises Tungsten.

19. An intravascular catheter as in claim 16, wherein the reinforcement layer comprises a braid.

20. An intravascular catheter as in claim 19, wherein the braid comprises multiple stands.

21. An intravascular catheter as in claim 20, wherein the multiple strands are braided in a three-over-three pattern.

22. An intravascular catheter as in claim 20, wherein the multiple strands are braided in a four-over-four pattern.

23. An intravascular catheter as in claim 20, wherein the multiple strands are braided in a five-over-five pattern.

24. An intravascular catheter as in claim 19, wherein the elongate shaft further comprises an inner layer and an outer layer with the reinforcement layer disposed therebetween.

25. An intravascular catheter as in claim 24, wherein the elongate shaft further comprises a soft distal tip.

26. An intravascular catheter as in claim 25, wherein the inner layer, the outer layer and the reinforcement layer extend from a proximal end of the shaft to a proximal end of the distal tip.

27. An intravascular catheter as in claim 26, wherein the reinforcement layer comprises a plurality of non-magnetically responsive radiopaque metal wires.

28. An intravascular catheter as in claim 27, wherein the outer layer comprises a non-radiopaque polymer.

29. An intravascular catheter as in claim 28, wherein the outer layer is non-radiopaque.

30. An intravascular catheter as in claim 29, wherein the inner layer comprises a non-radiopaque lubricious polymer.

31. An intravascular catheter as in claim 30, wherein the distal tip comprises a polymer loaded with a radiopaque non-magnetically responsive filler.

32. A method of using an intravascular catheter, comprising:
providing an intravascular catheter comprising an elongate shaft that is entirely non-magnetically responsive and at least partially radiopaque, the elongate shaft including a reinforcement layer comprising a non-magnetically responsive radiopaque metal;
inserting the catheter into a patient's vascular system; and
utilizing MRI to visualize the catheter in the vascular system.
